(19) United States
(12) Patent Application Publication
(10) Pub. No.: US 2024/0399124 A1
(43) Pub. Date: Dec. 5, 2024

(54) VAGINAL DILATOR WITH AUTOMATED EXPANSION SYSTEM AND TELEMEDICINE SYSTEM

(71) Applicant: THE REGENTS OF THE UNIVERSITY OF CALIFORNIA, Oakland, CA (US)

(72) Inventors: Rafaela SIMOES-TORIGOE, La Jolla, CA (US); Jyoti MAYADEV, La Jolla, CA (US); Milan MAKALE, La Jolla, CA (US); Karcher MORRIS, La Jolla, CA (US); Matthew KOHANFARS, La Jolla, CA (US); Po-Han CHEN, La Jolla, CA (US); Shengfan HU, La Jolla, CA (US); Yu LI, La Jolla, CA (US); Casey WILLIAMSON, La Jolla, CA (US); Frank TALKE, La Jolla, CA (US)

(21) Appl. No.: 18/698,758

(22) PCT Filed: Oct. 19, 2022
(86) PCT No.: PCT/US2022/047078

§ 371 Date: Apr. 4, 2024

Related U.S. Application Data
(60) Provisional application No. 63/257,285, filed on Oct. 19, 2021.

(51) Int. Cl. A61M 29/02; A61H 19/00; A61H 23/02

(52) U.S. Cl. CPC A61M 29/02; A61H 19/44; A61H 23/02; A61H 2201/0103; A61H 2205/087; A61M 2205/0238; A61M 2205/3327; A61M 2205/3344; A61M 2205/3368; A61M 2205/3379; A61M 2205/3553; A61M 2205/36; A61M 2210/1475

(57) ABSTRACT

An expandable vaginal dilator includes a stretchable sheath defining at least one fluid chamber therein and being shaped and sized according to vaginal measurements and physical examination. A support within the sheath seals and holds the sheath and provides one or more fluid channels configured for delivery of fluid into the at least one fluid chamber to expand the sheath. The support is configured to permit vaginal insertion of the sheath when the sheath is in an unexpanded state and to stabilize the sheath during expansion of the sheath. One or both of the sheath and support can be sized and shaped according to the physical exam of a patient. One or both of the sheath and support can be configured to provide differential expansion according to the physical patient information and a treatment plan.

### PRIORITY CLAIM AND REFERENCE TO RELATED APPLICATION

The application claims priority under 35 U.S.C. § 119 and all applicable statutes and treaties from prior U.S. provisional application Ser. No. 63/257,285 which was filed Oct. 19, 2021.

### FIELD

A field of the invention is medical devices. Another field of the invention is telemedicine. The invention concerns a vaginal dilator for prevention and treatment of vaginal stenosis (VS). Other applications include testing of gynecologic cancer survivors, and other gynecological conditions such as vaginal stenosis, vaginismus, pelvic floor training, recovery after vaginal reconstruction, etc.

### BACKGROUND

Cervical and vaginal dilators have been pursued over many decades as a way of expanding the vagina under various circumstances such as childbirth and stenosis. For example, Michaels in 1980 [U.S. Pat. No. 4,237,893] described a cervical dilator which swells once fluid enters a flexible polymer laminate. Cowan in 1997 [U.S. Pat. No. 5,947,991] described a device that relies on a catheter-like balloon to expand the cervix to facilitate labor. Ochiai in 1975 [U.S. Pat. No. 4,018,230A] described a mushroom shaped cervical dilator to assist with birth.

These dilator devices are small in size and intended for cervical placement making them difficult to be placed by the patient themselves. Moreover, they are not designed to provide uniform and incremental pressure along the length of the vaginal vault and they do not include feedback sensors and warmth to expand collagen scars.

Hakim et al., in 2016 [WO 2016/167996 A1] describe a detachable vaginal dilator to treat stenosis. The device is very complicated to manufacture, difficult for patients to use, and does not include an automated pump and sensor feedback system to incrementally expand the vaginal vault. Owing to its large dimensions would also be difficult to insert in patients with advanced stage stenosis. Courtion et al [US Patent WO2015070242A2] introduced a system for vaginal wall rejuvenation through the use of light and vibration without the customizable geometries, expansion capabilities, and wireless monitoring required in a patient population suffering from VS.

Lamoureux & Diaz [WO2016040610] describe a vaginal dilator having an elongate flexible shaft and a soft distal tip. This catheter like device includes an inflation lumen in the shaft and tip. The dilator is designed for use by a doctor or other medical professional and is not suited for self-use by a patient or by a patient with telemedical assistance. Similarly, [CN111671387] describes adjustable inflatable vaginal dilator for obstetrics and gynecology departments.

Conventional home use vaginal dilators are often prescribed by doctors to decrease anxiety and pain in anticipation of dyspareunia (genital pain during intercourse) or vaginal examinations [Liu, M., Juravic, M., Mazza, G., and Krychman, M. L., 2021, “Vaginal Dilators: Issues and Answers,” Sexual Medicine Reviews, 9 (2), pp. 212-220]. Vaginal dilators are typically smooth, cylindrical devices that are inserted into a woman's vagina to facilitate the stretching and relaxation of the underlying tissues [Juravic et al., supra]. The use of vaginal dilators can promote epithelialization and increased vascularity of the tissues after radiation treatment [Hartman, P., and Diddle, A. W., 1972, “Vaginal Stenosis Following Irradiation Therapy for Carcinoma of the Cervix Uteri,” Cancer, 30 (2), pp. 426-429]. These vaginal dilators are commonly sold in multiple sizes [Miles, K., and Miles, S., 2021, “Low Dose, High Frequency Movement Based Dilator Therapy for Dyspareunia: Retrospective Analysis of 26 Cases,” Sexual Medicine, 9 (3), p. 100344]. They are typically made of hard plastic or latex materials. Some dilator models have additional features to improve user experiences, such as temperature or vibration control. For instance, dilators that feature an auto-heated, vibrating design help decrease pain sensitivity and dilator handles make it easier to insert and hold the dilator in place [Juravic et al., supra].

A vaginal insert with mechanical hand controls is difficult for a user and is likely to cause the discomfort of typical dilators discussed above. [CN 104689459]. This insert is intended for use in childbirth and provide the type of dilation associated with childbirth. It is not suited for treatment of vaginal stenosis.

### SUMMARY OF THE INVENTION

A preferred embodiment provides an expandable vaginal dilator that includes a stretchable sheath defining at least one fluid chamber therein and being shaped and sized according to vaginal measurements. A support within the sheath seals and holds the sheath and provides one or more fluid channels configured for delivery of fluid into the at least one fluid chamber to expand the sheath. The support is configured to permit vaginal insertion of the sheath when the sheath is in an unexpanded state and to stabilize the sheath during expansion of the sheath. One or both of the sheath and support can be sized and shaped according to the 3D measurements scan of a patient. One or both of the sheath and support can be configured to provide differential expansion according to the 3D patient information and a treatment plan.

A vaginal dilator system can include a bidirectional peristaltic pump in fluidic communication with an expansion fluid reservoir and the support to controllably supply expansion fluid into the at least one fluid chamber, and optionally support a second chamber. Pressure, volume and/or strain sensors monitor expansion of the at least one fluid chamber. A feedback system adjusts the expansion according to pressure measurements provide by pressure, volume, and/or strain sensing.

### BRIEF DESCRIPTION OF THE DRAWINGS

FIG. 1 is a cross-sectional schematic view of a preferred embodiment vaginal dilator;

FIG. 2 shows a preferred automated vaginal dilator control system to control the FIG. 1 preferred embodiment vaginal dilator;

FIG. 3 shows a model of a preferred embodiment vaginal dilator used for finite element simulation;

FIGS. 4A and 4B are data showing relationships between the (A) pressure and dilator expansion (i.e. cross-sectional area), and between the (B) pressure and applied load on a flat surface for an experimental vaginal dilator;

FIGS. 5A-5C show simulated data from (A) von Mises Stress during expansion, (B) dilator pressure and expansion, as well as (C) pressure and applied load against rigid flat vaginal phantom surfaces;

FIG. 6 is an image of another complete protype system and vaginal dilator that was tested;

FIGS. 7A and 7B respectively show exemplary mold dimensions for a single chamber vaginal dilator and an insertion rod;

FIGS. 8A and 8B are respective cross-sectional schematic view of a preferred embodiment vaginal dilator and perspective view of a tip support of the vaginal dilator.

### DETAILED DESCRIPTION OF THE PREFERRED EMBODIMENTS

An embodiment of the invention is an expandable, customizable, personalized vaginal dilator. The dilator has a stretchable silicone sheath that can be manufactured through molding and casting and can optionally define multiple chambers therein. A support, preferably in the form of an inner rod provides stabilization and includes fluid channels for air/water, allowing the dilator to have a small and comfortable insertion diameter. The support connects to a fluid source and provide stabilization for insertion. The dilator is activated by an automatic expansion system including a bidirectional peristaltic pump attached to the expandable dilator. A driver provides for expansion of the dilator, and a feedback system adjusts the expansion according to pressure measurements provide by pressure/volume sensing. A reservoir is connected to the housing unit that supplies the expandable dilator device with expansion fluid. The dilator will expand with the input of the fluid causing an outward directed pressure. The fluid is preferably temperature regulated. The sheath/rod structure can include domes or other custom vaginal shapes that can be arranged to provide for predetermined amounts of expansion in predetermined directions or locations and different amounts in other locations. The sheath/rod structure can be personalized, shaped and dimensioned according to patient measurements.

Sensors used can include mechanical strain gauge sensors built into the pressurization system outside the body that monitor resistance pressure of the vaginal wall so that expansion is gradual. These data are stored in a controller and downloaded to the clinic via cell phone linkage, allowing for a telemedicine paradigm. This telemedicine capability will facilitate the assessment of progress and monitoring of patient home usage patterns of the device. A prominent shut-off button can be included for patient use in response to any discomfort during use. A temperature limiting system preferably is included to prevent expansion when an expansion fluid, e.g. saline, exceeds a predetermined temperature, e.g. when temperature is above 39° C. and the system saline pump will not engage at 40° C. Preferably, the system fluid is heated to a temperature that promotes blood flow and healing of damaged tissues, which is a temperature of 38° C.

Generally, preferred dilators include features for controlled and compliant expansion and can be pressurized to maximize patient comfort. The expansion of the dilator should be personalized, with gradual expansion based on patient specific vaginal dimensions, to decrease the initial force being applied to the vaginal wall and thus improve tolerability and patience adherence. This avoids a problem with a set expansion, which can cause pain during use because conventional devices tend to assert a maximum load for the smallest vaginal widths, which is a critical issue in vaginal stenosis treatment. With a dilator and system of the invention, the expandable design coupled with pressure measurements indicating expansion and the applied load against the vaginal wall can increase patient adherence by providing a graded better tolerated therapy.

Preferred dilators are personalized, sized and shaped according to patient measurements. Because the silicon sheath and its insertion rod can readily be fabricated based upon such measurements, or a dilator can be selected from a plurality of pre-made dilators with different dimensions and/or shape. Size and shape selection can be made based upon the vaginal length, and width at the apex, mid and distal vagina.

A heating element can be used to heat fluid used to expand the dilator. A vibration motor can provide for controlled vibration of the dilator. The device additionally can change frequency of vibration automatically or via control from a user interface, such as a knob on the housing unit.

Another optional feature is a coating on the sheath. The coating, for example can provide therapeutic materials, or can include a scaffold or biomaterial used for promoting vaginal healing from radiation damage/side effects. A therapeutic coating can include a medication. Preferred therapeutic coatings include an estrogen creams, anti-inflammatoires, topical cytokines, a topical steroid creams, vascular endothelial growth factors (VEGF), or other medication targeted options.

A preferred dilator system includes a heating element, a fluid reservoir, a vibration control interface, such as a knob, a pressure/expansion progress display, a temperature control interface, a system control interface, a thermally insulated connector. The expandable dilator section preferably includes an expandable silicone sheath that can be controlled to expand to a plurality of different shapes.

A telemedicine system includes communications with the feedback system and data storage of pressure, volume changes of multi-chambered dilator. The system can also provide some data driven clinical interpretations for feedback to a patient and a medical practitioner. A user interface is designed to encourage patient compliance with planned testing/monitoring/treatment plans. This component allows patient at home care delivery with telehealth monitoring with the health staff and physicians for oversight.

The user interface can be used by patient to provide patient controlled and/or programmatically running cycles of expansion/dilation based user/sensor feedback.

Preferred embodiments of the invention will now be discussed with respect to experiments and drawings. Broader aspects of the invention will be understood by artisans in view of the general knowledge in the art and the description of the experiments that follows.

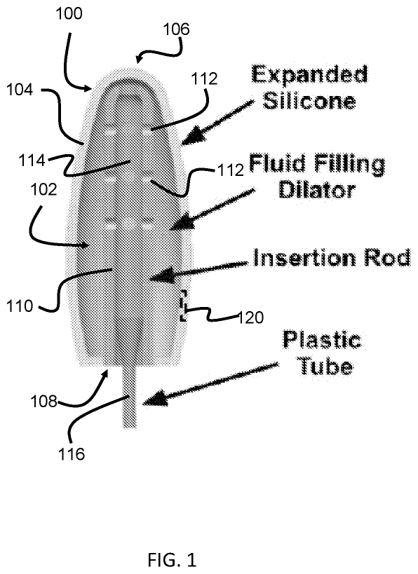

FIG. 1 shows a preferred embodiment vaginal dilator 100 that was made and tested as a prototype. The vaginal dilator 100 is show in an expanded state, which is achieved via fluid pressure introduced into a fluid chamber 102 defined by a stretchable silicone sheath 104. The sheath 104, when inflated, is shaped and sized according to vaginal measurements. In particular, the sheath 104 is preferably shaped, structured and sized in 3D according to 3D measurements of a patient's vaginal canal and according to a treatment plan for that patient. A preferred sheath 104 can also provide patient specific differential expansion. To achieve differential expansion a wall of the sheath can vary in thickness or in shape. Having a non-uniform shape or thickness can direct differential expansion to very specific parts of a patient's vaginal canal according to the 3D measurements and treatment plans. 3D vaginal measurements such as CT can provide for very specific personalized dilator. Manual measurements can also contribute to personalization, but the use of 3D CT scans allows for very precise size and shape specificity. The thickness in the sheath 104 can vary in one or moth of the longitudinal and circumferential directions along the sheath to achieve patient specificity.

The sheath 104 has a closed distal end 106 and a (sealed) opening 108 at its proximal end. The opening 108 is sealed to a support in the form of an inner insertion rod 110 that is coaxial and centrally located within the sheath 104. The insertion rod 110 can also be shaped in 3D according to patient 3D measurements, which can contribute to differential expansion of the sheath 104. The support provides stabilization and some rigidity to allow comfortable insertion when the fluid chamber 102 is an unexpanded state. The insertion rod 110 stabilizes and seals the fluid chamber 102 at the opening 108. The insertion rod 110 includes a plurality of fluid channels/ports 112 arranged to introduce expansion fluid in the fluid chamber 102. The insertion rod 110 includes a central lumen 114 for transporting fluid, to the channels 112, which lumen connects to an external fluid source via a tube 116. The tube 116 is preferably detachable from the vaginal dilator 100. One or more sensors 120 can be embedded in the sheath, such as a strain, temperature or motion sensor and provide information for a control system via wired or wireless connection.

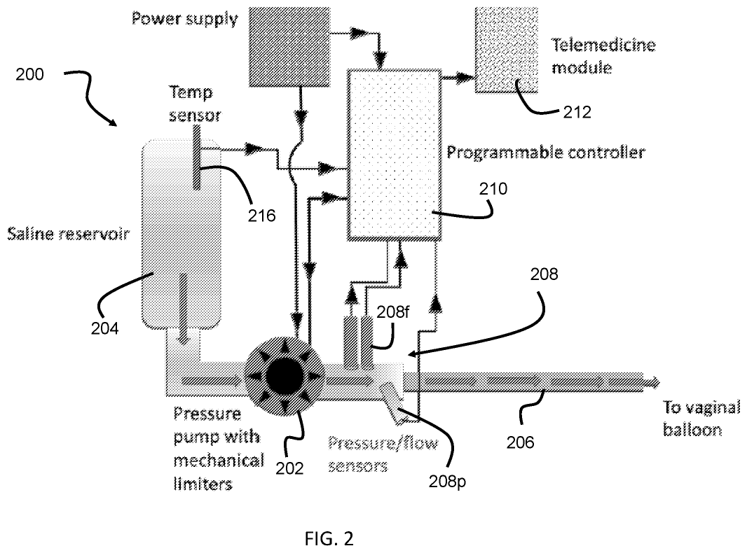

FIG. 2 shows a preferred automated vaginal dilator control system 200 for use with the vaginal dilator 100. The control system 200 includes a bidirectional peristaltic fluid pump 202 attached to an expansion fluid reservoir 204 and sends fluid (which can be liquid or gas, such as air) to the insertion rod 110 via a tube 206 to controllably supply expansion fluid into the fluid chamber 102. In one embodiment, the tube 206 includes an end that forms the tube 116 for attachment to the dilator 100. One or more sensor 208 monitor pressure, volume or strain to monitor expansion of the fluid chamber. The sensor(s) 208 are indirect in FIG. 2, monitoring flow and/or pressure, from which a controller 110 can calculate expansion of the fluid chamber 102 and use the monitored expansion to control the fluid pump 202. The sensors 208 could also include or consist of a strain sensor attached to or within the sheath 104 that has a wired or wireless connection to the controller 210, which can also calculate expansion of the fluid chamber 102 from strain of the sheath 104. In FIG. 2, a flow sensor 208f and pressure sensor 208p are shown. A feedback system including the controller 210 can also include a telemedicine module 212 for communications with health professionals for monitoring and or providing additional control of the dilator 100 through the controller 210. The telemedicine module 212 can be, for example, an app on a smart phone that communicates with the controller. A temperature sensor 216 monitors fluid (such as saline) in the reservoir 204 and the sensor 216 can also include a heating element to maintain a desired predetermined temperature.

In preferred embodiment, the controller 210 is preprogrammed to automatically fill with saline from the reservoir 204 to ensure proper operation and expansion on successive days when used by a patient. The dilator is mechanically limited by the design of the sheath 104 for each patient's vaginal volume to avoid exertion of too much pressure on the vaginal wall. The system preferably includes a shutoff button, such as on a power supply 218 (or on the pump 202 or controller 210), so if patients are uncomfortable the system turn off and the saline will immediately drain out back into the reservoir 204. The controller 210 preferably sets a warm saline temperature to 38° C. to promote blood flow and healing. The controller preferably limits temperature to a maximum of 40° C., and will not allow the system saline pump 202 to expand the dilator 100 when temperature reaches 40° C. The tube insertion rod 110 can also include a vibrator motor and direct electric or wireless connection to the controller, as vibration therapy can also help in therapy.

An experimental device consistent with the vaginal dilator 100 was tested. The prototype design includes a three-part mold with a removable insertion rod. The mold consisted of two parts that can be attached and a solid insertion rod that fits in the middle of the mold. Silicone sheaths of various wall thicknesses were created to determine the ideal wall thickness.

Smooth-on medical grade RTV (room temperature vulcanizing) silicone was poured in a dilator mold. After the silicone was cured, both the inner and outer molds were removed. A plastic tube was inserted into the 3D printed inner rod containing air channels to enable the inflation of the dilator. The air channel rod was then placed inside the silicone sleeve for inflation and to stabilize dilator shape. Following dilator assembly, the gap between the air channel rod and the silicone sleeve was sealed with silicone and left to cure. To expand the dilator, the plastic tube can be attached to any type of fluid pump (which can be liquid or gas, such as air). Prototype devices were tested with vaginal phantoms.

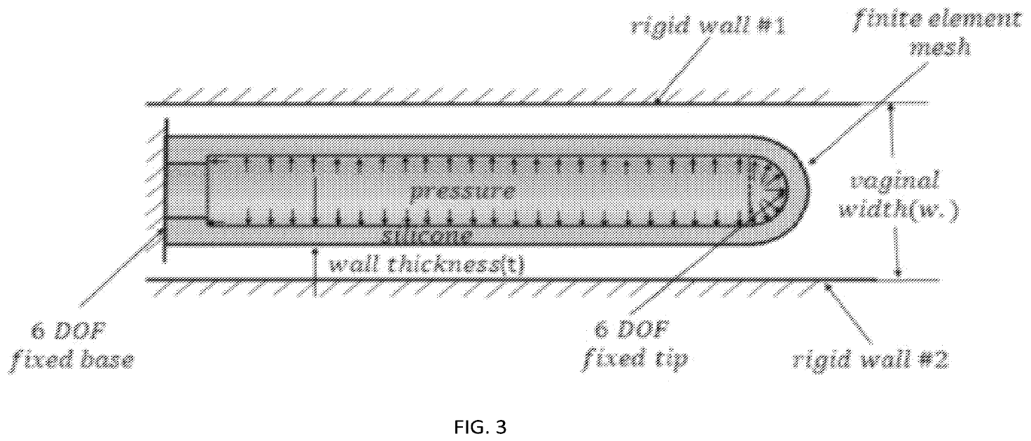

A finite element model shown in FIG. 3 was developed using Altair® Hypermesh® to complement the experimental results for both dilator expansion and the applied load against flat surfaces acting as simplified vaginal wall phantoms. The nominal model consisted of an expandable dilator sheath with a wall thickness of 2.5 mm, a core diameter of 11.5 mm, and a length, from base to tip, of 76 mm. Approximately 30,000 4-node tetrahedron elements were used to mesh the model. Mooney-Rivlin hyperelastic material models were implemented for comparison and a final model was chosen by fitting the expansion experimental results to the finite element simulation output, similar to the approach proposed by Gopesh et al. T. Gopesh and J. Friend, “Facile Analytical Extraction of the Hyperelastic Constants for the Two-Parameter Mooney-Rivlin Model from Experiments on Soft Polymers,” Soft Robotics, p. soro.2019.0123, (2020). The best fit Mooney-Rivlin coefficients were found to be C₀₁=70 kPa and C₁₀=0.258 kPa.

Pressure was ramped up from 0 mmHg to 362 mmHg and the dilator expanded freely until it was constrained by the two parallel walls simulating the vaginal walls (separated by a nominal distance of 18 mm). The numerical results of dilator expansion (maximum cross section area) versus pressure and force against the vaginal walls versus pressure were calculated using LS-DYNA®, which is an explicit transient finite element solver.

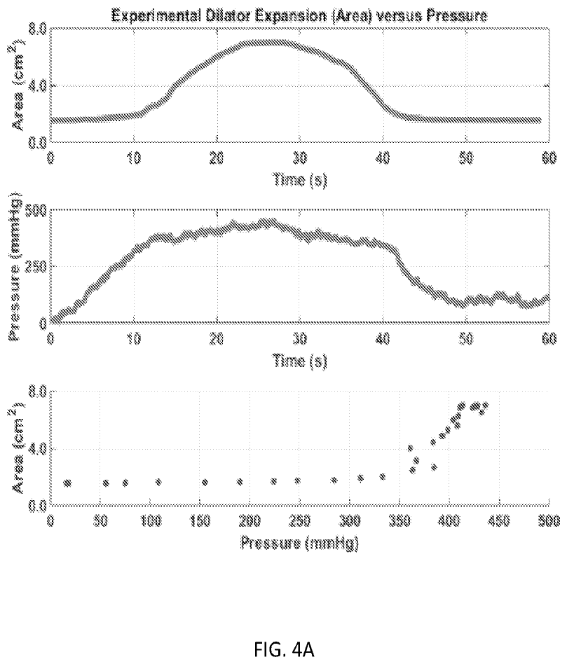

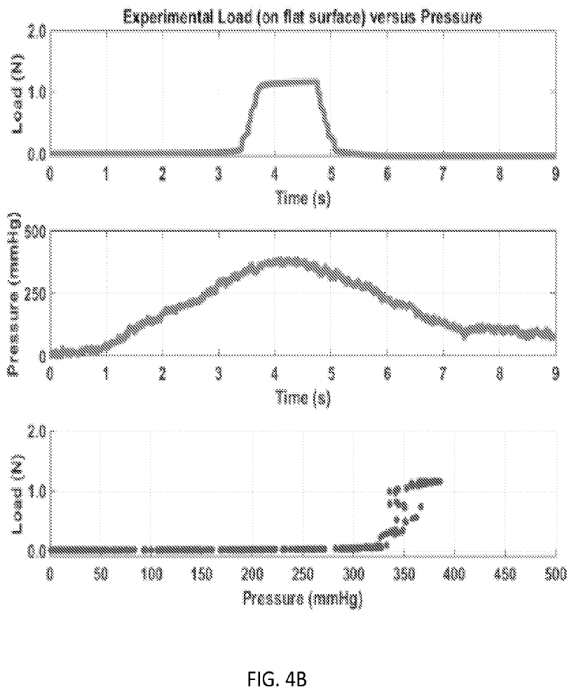

The relationships between the (A) pressure and dilator expansion (i.e. cross-sectional area), as well as between the (B) pressure and applied load on the flat surface were obtained and shown in FIGS. 4A and 4B. As a 60 cc syringe pumped air into and out of the silicone dilator, the internal pressure was observed to increase and decrease, as expected. For case (A), where the flat surfaces are not implemented, the cross-sectional area was found to increase and decrease in response to the pressurization. At approximately 310 mmHg, the dilator began to rapidly expand in relation to pressure (FIG. 4A). The maximum cross-sectional area recorded was 6.5 cm² at a pressure of 440 mmHg, representing an increase in area of more than 400 percent.

In FIG. 4B, where the flat surfaces were positioned with a spacing of 18 mm, the applied load against one surface was shown to increase and decrease in response to pressurization only above the 310 mmHg threshold, when the dilator was rapidly expanding. The maximum load against the flat surface was observed to be 1.2 N.

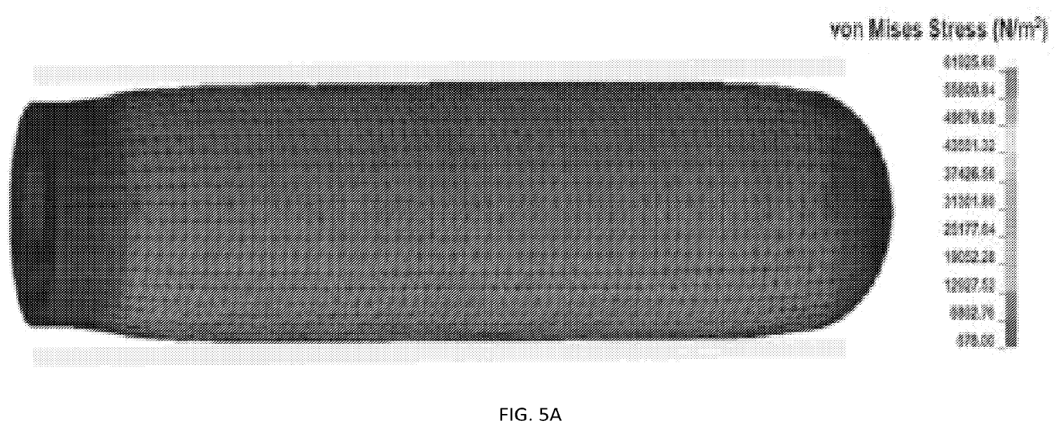

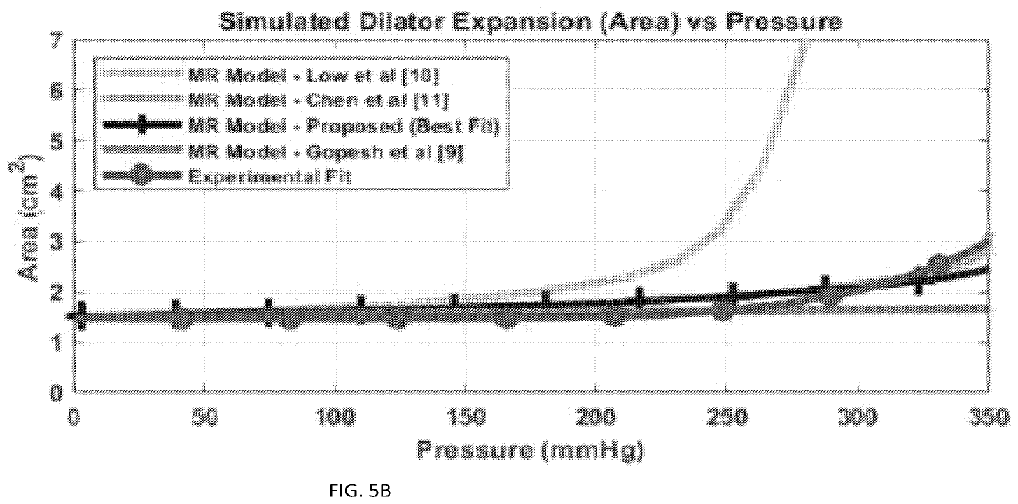

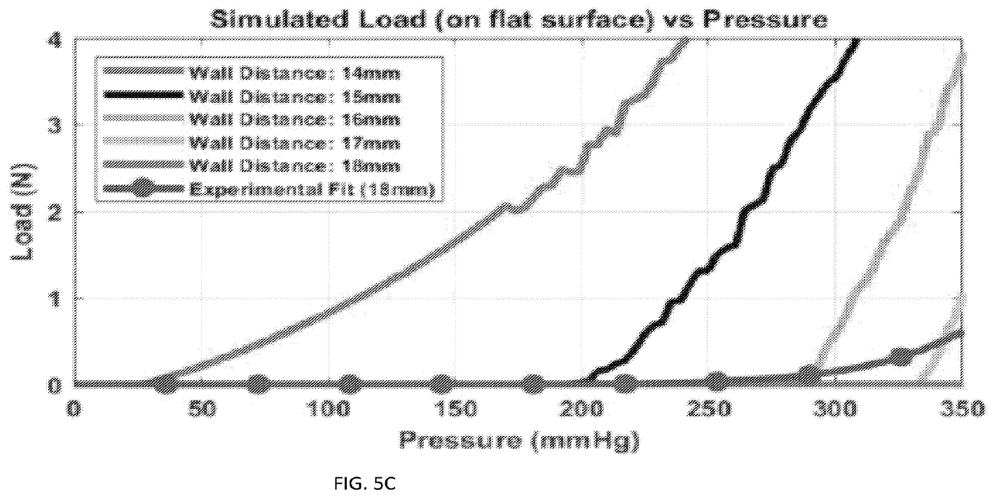

Finite element simulations complement the experimental study by similarly exploring the relationships between (A) von Mises Stress during expansion, (B) dilator pressure and expansion, as well as (C) pressure and applied load against rigid flat surfaces. FIG. 5A shows the von Mises Stress distribution as vaginal dilator is being expanded and FIG. 5B shows results for simulated expansion with respect to pressure for each of the material models used. The material models were from [9] T. Gopesh and J. Friend, “Facile Analytical Extraction of the Hyperelastic Constants for the Two-Parameter Mooney-Rivlin Model from Experiments on Soft Polymers,” Soft Robotics, p. soro.2019.0123 July 2020; J.-H. Low, M. H. Ang, and C.-H. Yeow, “Customizable soft pneumatic finger actuators for hand orthotic and prosthetic applications,” in 2015 IEEE International Conference on Rehabilitation Robotics (ICORR), Singapore, Singapore, August 2015, pp. 380-385; Z. Chen, S. Wang, C. Zhang, P. Zhang, and Z. Liao, “Anthropomorphic flexible joint design and simulation,” in 2020 15th IEEE Conference on Industrial Electronics and Applications (ICIEA), Kristiansand, Norway, November 2020, pp. 1673-1678. The experimental data is also shown for the same region of pressurization.

An improved material model was implemented based on experimental results obtained in this model in order to conduct further numerical simulations. FIG. 5C shows results for the improved model for load against the rigid flat surfaces as the dilator is pressurized. The distance between the flat surfaces simulating vaginal walls are varied from 18 mm, which was the spacing used in the experimental setup, to 14 mm, where the spacing between walls corresponded to the diameter of the dilator cross-section. As the wall separation distance decreased from 18 mm to 14 mm, the pressure threshold to measure a significant load applied to the simulated vaginal wall decreased, while the overall maximum applied load increased. The experimental results for an 18 mm wall diameter are included in FIG. 5C. The results vary slightly from the simulated results for an 18 mm wall distance; however, they are consistent with the trends exhibited by the finite element simulations. According to the simulation, the applied load depends on both internal dilator pressure and wall distance.

As wall distance decreases, the maximum load that can be applied by the dilator on the simulated vaginal walls increases. This suggests that as vaginal width decreases, a set expansion of the vaginal dilator would lead to an increased load in the vaginal walls, which would likely lead to a painful experience when using the device. Therefore, the expansion of the dilator should be personalized and controlled, with gradual expansion based on patient specific vaginal dimensions, to decrease the initial force being applied to the vaginal wall and thus improve tolerability and patience adherence.

The experiments showed that a vaginal dilator of the invention can provide effective treatment and prevention of radiation-induced VS provides a gradual expansion mechanism to mechanically expand the vaginal canal. This can apply a more uniform, and potentially less painful and injurious, load across the vaginal wall than is possible with manual rod-shaped dilators.

The relationships among pressure, expansion, and applied load determined via the experiments described above provide controlled methods to increase device efficacy at preventing, treating, and monitoring VS progression. The expansion of the proposed vaginal dilator can be estimated from the results portraying cross-sectional area as a function of pressure as in FIG. 5A. This relationship can be used to track patient progress via the controller and a telemedicine module/app by monitoring the changes in dilator pressure measurements, which indicate the resistance of the vaginal wall due to fibrotic scarring. As the scar tissue dissociates, the resistance of the wall related to pressure is expected to decline. This allows further data on vaginal stenosis to be recorded, giving the opportunity for this condition to be further understood and treated. Progress tracking can also serve as motivation for patients, which can improve patient adherence.

The present expandable dilator design coupled with pressure measurements indicating expansion and the applied load against the vaginal wall can increase patient adherence by providing a graded better tolerated therapy. The present automated system that can analyze patient compliance and provide data to the closed-loop feedback control system for expansion for optimal comfort and therapeutic effect.

3D printing can also be used, and in additional experiments designed, 3D protypes were printed, and instrumented with vibration and heating elements. 3D printing and coating with silicone renders the dilator surface highly biocompatible. Devices can be 3D printed from a patient MRI or CT scan of the inflated and contrast filled vaginal vault. This allows the creation of a personalized patient insert at very low cost.

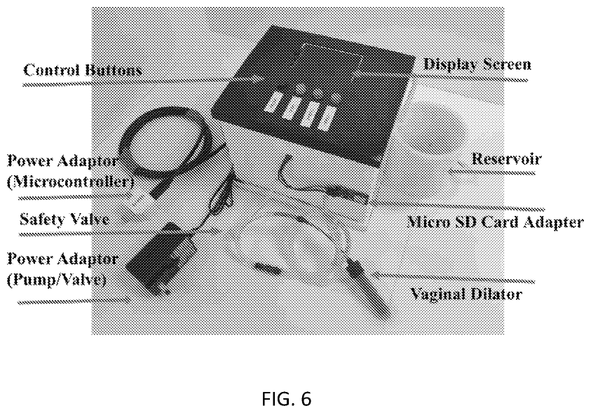

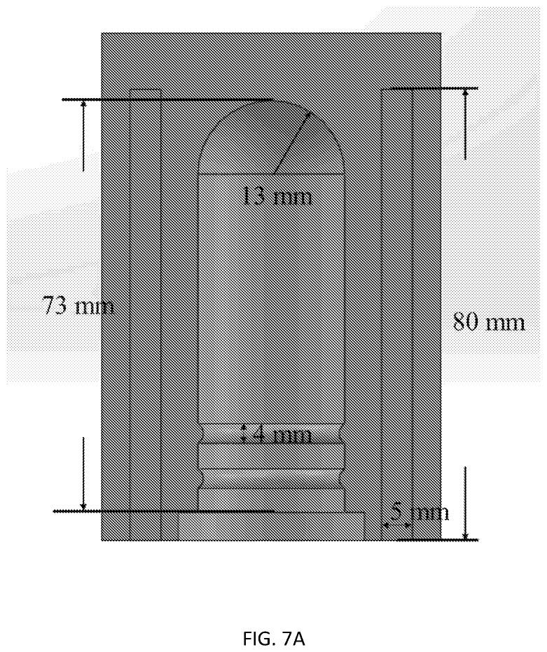

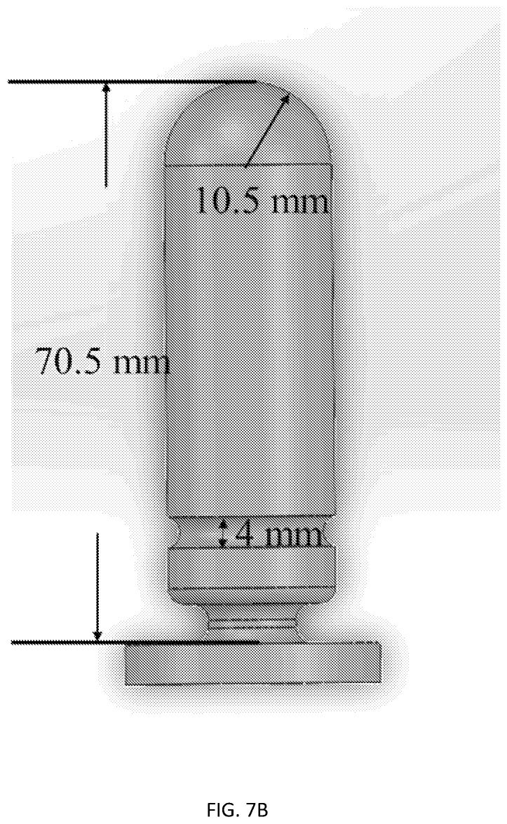

FIG. 6 is an image of another complete protype system 200 and vaginal dilator 100 that was tested. FIGS. 7A and 7B respectively show exemplary mold dimensions for a single chamber vaginal dilator and an insertion rod. Preferred steps of using the dilator and the monitoring system to perform vaginal dilation therapy are as follows. 1) Connect the dilator to the monitor system. 2) Connect the power adapters to a power supply, and check that the monitor lights up. Make sure there is a micro-SD card in the card reader slot to store the data. 3) Pour warm saline solution into the reservoir. 4) Insert the vaginal dilator to the vagina with lubricant. 5) Choose a suitable therapy mode and start the therapy. For patients that have more advanced vaginal stenosis, a therapy mode where the dilator does not expand too much or too fast will be more suitable, with the opposite applying to less severe cases of vaginal stenosis.

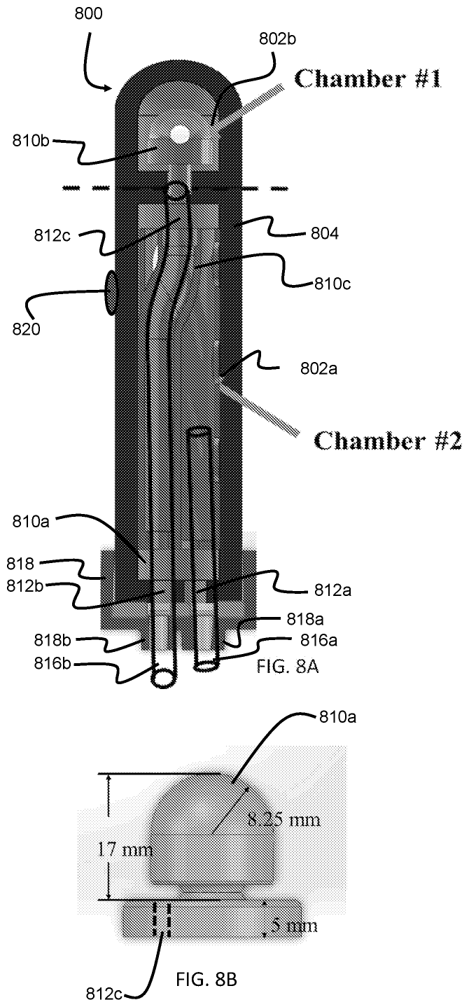

A dual chamber vaginal dilator 800 was also formed and is shown in FIGS. 8A and 8B. The vaginal dilator 800 is show in an unexpanded state and has two separate a fluid chambers 802a and 802b defined by a stretchable silicone sheath 804 and an insertion rod tip 810b (see also FIG. 8B). A base 810a is a separate part that forms a two-piece insertion rod with the tip 810b that together perform the basic function of the support/insertion rod 110, with a connecting portion 810c. The connection portion 810c provides structure from the base 810a and its two ports 812a and 812b that secure tubes 816a and 816b for introducing expansion fluid in the fluid chambers 802a and 802b. A third port 812c also accepts the tube 812b to bring fluid into the fluid chamber 802b. The connecting portion 810c includes a lumen that accepts and routes the tube 816b into the upper chamber 802b. A separate lower base 818 serves to seal the sheath 804 and also provides ports 818a and 818b for the tubes 812a and 812b. A dome or other shaped structure 820 is shown. The sheath can include many such shaped structures, that can be determined according to the imaging or physical examination of the particular vaginal canal of the patient and a dilation treatment plan. The two fluid chambers 802a and 802b can be expanded independently and by different amounts.

The dual chamber dilator 800 (or multi-chamber with more than two chambers) provides for differential inflation that, for example, can dilate the apex part of the vaginal canal close to the cervix. Such a dual or multi chamber dilator can also be designed to focus on dilating a certain area of the vaginal canal that has severe vaginal stenosis, according to patient specific imaging.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. An expandable vaginal dilator, comprising:
a stretchable sheath defining at least one fluid chamber therein and being shaped and sized according to vaginal measurements; and
a support within the sheath that seals and holds the sheath and provides one or more fluid channels configured for delivery of fluid into the at least one fluid chamber to expand the sheath, wherein the support is configured to permit vaginal insertion of the sheath when the sheath is in an unexpanded state and to stabilize the sheath during expansion of the sheath.

2. The expandable vaginal dilator of claim 1, wherein the sheath and support are sized and shaped according to a personalized patient measurement.

3. The expandable vaginal dilator of claim 2, wherein one or both of the sheath and support are sized and shaped according to a 3D measurement scan of a patient.

4. The expandable vaginal dilator of claim 1, wherein the sheath is structured to provide differential expansion.

5. The expandable vaginal dilator of claim 4, wherein the differential expansion is set according to the 3D scan of the patient.

6. The expandable vaginal dilator of claim 4, wherein the differential expansion is obtained according to thickness changes of the sheath in the longitudinal and circumferential directions along the sheath.

7. The expandable vaginal dilator of claim 1, wherein the sheath is formed of silicone.

8. The expandable vaginal dilator of claim 1, wherein the sheath comprises a therapeutic coating.

9. The expandable vaginal dilator of claim 8, wherein the therapeutic coating comprises a medication.

10. The expandable vaginal dilator of claim 9, wherein the medication is selected from the group consisting of an estrogen cream, an anti-inflammatory, a topical cytokine, a topical steroid cream, and a vascular endothelial growth factor (VEGF).

11. The expandable vaginal dilator of claim 1, wherein the sheath comprises a strain sensor.

12. The expandable vaginal dilator of claim 1, wherein the sheath comprises a motion sensor.

13. The expandable vaginal dilator of claim 1, wherein the at least one fluid chamber comprises at least two independent fluid chambers.

14. The expandable vaginal dilator of claim 13, wherein the two independent fluid chambers comprise a tip chamber and a base chamber, and the support comprises a separate tip and base support.

15. The expandable vaginal dilator of claim 1, wherein the support comprises an inner rod coaxial and within the sheath that stabilizes and seals the at least one fluid chamber at a proximal end opening, of the sheath, the rod including a plurality of fluid channels arranged to introduce expansion fluid in the at least one fluid chamber.

16. The expandable vaginal dilator of claim 1, comprising one or more shaped structures arranged on the sheath to provide for predetermined amounts of expansion in predetermined directions or locations and different amounts in other locations.

17. The expandable vaginal dilator of claim 16, wherein the one or more shaped structures are located and sized according to a vaginal physical exam to conform to the vaginal canal of the patient and a dilation treatment plan.

18. The expandable vaginal dilator of claim 1, comprising a vibration motor.

19. A vaginal dilator system including an expandable vaginal dilator of any previous claim, the system comprising:
a bidirectional peristaltic pump in fluidic communication with an expansion fluid reservoir and the support to controllably supply expansion fluid into the at least one fluid chamber;
pressure, volume and/or strain sensors to monitor expansion of the at least one fluid chamber; and
a feedback system that adjusts the expansion according to pressure measurements provide by pressure, volume, and/or strain sensing.

20. The vaginal dilator system according to claim 19, comprising a heater for the expansion fluid, wherein the feedback system regulates temperature of the expansion fluid.

21. The vaginal dilator system according to claim 20, wherein the temperature is regulated to be approximately 38° C.-39° C.

22. The vaginal dilator system according to claim 19, comprising a telemedicine communications module.
